(19) United States
(12) Patent Application Publication
(10) Pub. No.: US 2025/0049918 A1
(43) Pub. Date: Feb. 13, 2025

(54) TARGETING THE IMMUNE SUPPRESSIVE TUMOR MICROENVIRONMENT

(71) Applicant: BROWN UNIVERSITY, Providence, RI (US)

(72) Inventors: Wafik S. El-Deiry, Barrington, RI (US); Praveen Srinivasan, Providence, RI (US)

(21) Appl. No.: 18/472,445

(22) Filed: Sep. 22, 2023

Related U.S. Application Data
(60) Provisional application No. 63/408,901, filed on Sep. 22, 2022.

(51) Int. Cl. A61K 39/00

(52) U.S. Cl. CPC A61K 39/4621; A61K 39/4611

(57) ABSTRACT

An immune suppressor cell modified to include an innate immune effector is described. Methods of using the modified immune suppressor cell to treat cancer are also described.

### CROSS-REFERENCE TO RELATED APPLICATIONS

This application claims priority to U.S. Provisional Patent Application Ser. No. 63/408,901, filed Sep. 22, 2022, which is incorporated herein by reference.

### REFERENCE TO SEQUENCE LISTING XML

The instant application contains a Sequence Listing XML (Name: BU-031768-US-ORD-SeqListing-ST26.xml; size: 4,723 bytes; and Date of Creation: Jan. 22, 2024) which has been submitted in XML format via EFS-Web and is hereby incorporated by reference in its entirety.

### BACKGROUND

It is known that genetic and epigenetic changes occur in tissues as they transform to take on the phenotype of a cancer cell. Different steps in the malignant transformation process, including acquisition of the mutator phenotype, which is associated with loss of tumor suppressor activity, often results in the generation of neoantigens, which are subject to immune recognition.

Various attempts have been made to help the immune system to fight tumors. One early approach involved a general stimulation of the immune system through the administration of bacteria (live or killed) to elicit a general immune response which would also he directed against the tumor. Existing innate stimulators of immunity include BCG [Mukherjee et al., Curr Opin Urol 29 (3): 181-188 (2019)], lyophilized incubation mixture of group A Streptococcus pyogenes (OK-432) [Pan et al., Immunol Cell Biol, 92 (3): p. 263-74 (2014)], CSF-470 [Pampena et al., Front Immunol, 9: p. 2531 (2018)], as well as doses of chemotherapies that can selectively suppress Treg cells [Noordam et al., Oncoimmunology, 7 (12): p. c1474318 (2019)].

It is known that the usual lack of a powerful immune response to tumor associated antigens (TAAs) is due to a combination of factors. T cells have a key role in the immune response, which is mediated through antigen recognition by the T cell receptor (TCR), and they coordinate a balance between co-stimulatory and inhibitory signals known as immune checkpoints. These inhibitory signals function as natural suppressors of the immune system as an important mechanism for maintenance of self-tolerance and to protect tissues from damage when the immune system is responding to pathogenic infection. However, dysregulated immune suppression reduces what could otherwise be a helpful response by the body to avoid the development of tumors. Cytokines, other stimulatory molecules such as CpG (stimulating dendritic cells).

An immune suppressive tumor microenvironment prevents effective cancer suppression by the host immune system as well as by reducing the efficacy of cancer immunotherapy. The recruitment of immune suppressive cells into a tumor inhibits the adaptive immune system from eliminating cancer cells in a growing tumor. Immune suppressive cells that are recruited include regulatory T-cells and myeloid-derived suppressor cells.

### SUMMARY

Currently the T-regulatory cells traffic to tumors and suppress anti-tumor immune responses. The current approach uses the trafficking of the cells for tumor targeting as well as a method to deliver anti-tumor innate immune effectors such as TRAIL. Furthermore, the current approach involves transduction of an innate immune effector such as TRAIL into immune suppressive T-regulator cells that traffic to the tumor microenvironment. The innate immune effector is expected to enhance cell death of tumor cells alone or in combination with other drugs, immune checkpoint and cellular therapies. Overcoming this barrier by arming such cells with an innate immune effector such as TRAIL would improve anti-tumor immunity and boost the efficacy of cancer immunotherapy including through use of CAR-T cells, immune check point therapy and immune modulatory antibodies.

In one aspect, the invention provides an immune suppressor cell modified to include an innate immune effector. In some embodiments, the immune suppressor cell is genetically modified to include an innate immune effector. In further embodiments, the immune suppressor cell is a regulatory T-cell or a myeloid-derived suppressor cell.

In some embodiments, the innate immune effector is TRAIL. In further embodiments, the TRAIL comprises an amino acid sequence having 95% identity to SEQ ID NO: 1, while in yet further embodiments the immune suppressor cell has been transformed to include a nucleotide sequence having 95% identity to SEQ ID NO: 2 that is operably linked to a promoter.

Another aspect of the invention provides a method of treating cancer in a subject. The method includes administering an effective amount of an immune suppressor cell modified to include an innate immune effector to the subject. In some embodiments, the immune suppressor cells are genetically modified. In further embodiments, the immune suppressor cell is a regulatory T-cell or a myeloid-derived suppressor cell.

In some embodiments, the innate immune effector is TRAIL. In further embodiments, the TRAIL comprises an amino acid sequence having 95% identity to SEQ ID NO: 1. In further embodiments, the immune suppressor cell has been transformed to include a nucleotide sequence having 95% identity to SEQ ID NO: 2 that is operably linked to a promoter.

In some embodiments, the cancer is selected from the group of cancer types consisting of sarcoma, carcinoma, and lymphoma. In further embodiments, the cancer is a solid tumor cancer selected from the group consisting of breast, colon, bladder, prostate, and lung cancer. In additional embodiments, the cancer is a drug-resistant cancer.

In some embodiments, an additional type of cancer therapy is used to treat the subject. In further embodiments, the method of treating cancer further comprises immune checkpoint therapy or administration of antibodies. In additional embodiments, the modified immune suppressor cells are administered directly to the tumor by injection.

### BRIEF DESCRIPTION OF THE FIGURES

FIG. 1 shows the nucleotide sequence of human TRAIL cDNA (SEQ ID NO:2) and its amino acid sequence (SEQ ID NO:1). The “N” at nucleotide position 447 (in SEQ ID NO:2) is used to indicate the nucleotide base may be a “T” or “G”.

### DETAILED DESCRIPTION OF THE INVENTION

The present invention provides an immune suppressor cell modified to include an innate immune effector is described. Methods of using the modified immune suppressor cell to treat cancer are also provided.

### Definitions

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment also includes partial or total destruction or differentiation of the undesirable proliferating cells with minimal effects on normal cells. In accordance with the present invention, desired mechanisms of treatment at the cellular level include stimulation of differentiation in cancer and pre-cancer cells.

As used herein, the term “prevention” includes either preventing the onset of a clinically evident unwanted cell proliferation altogether or preventing the onset of a preclinically evident stage of unwanted rapid cell proliferation in individuals at risk. Also intended to be encompassed by this definition is the prevention of metastasis of malignant cells or to arrest or reverse the progression of malignant cells. This includes prophylactic treatment of those having an enhanced risk of developing precancers and cancers. An elevated risk represents an above-average risk that a subject will develop cancer, which can be determined, for example, through family history or the detection of genes causing a predisposition to developing cancer.

“Pharmaceutically acceptable” as used herein means that the compound or composition is suitable for administration to a subject to achieve the treatments described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect, such as the inhibition of cancer growth by a detectable amount.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). In some embodiments, the subject is a human.

As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells that do not contain a recombinantly introduced nucleic acid.

As used herein, the term “cytokine” refers to a small protein (˜5-20 kDa) that is important in cell signaling, and in particular immunomodulation. Examples of cytokines include chemokines, interferons, interleukins, lymphokines, and tumor necrosis factors.

The inventors have determined that modifying immune suppressive cells that are recruited into tumors by arming the cells with an innate immune effector such as TRAIL provides an anticancer effect and improves the efficacy of cancer immunotherapy.

### Modified Immune Suppressor Cells

In one aspect, the present invention provides an immune suppressor cell modified to include an innate immune effector. The immune suppressor cells can be modified to release the innate immune effector, and/or they can be modified to express the innate immune effector bound to the surface of the immune suppressor cell. In some embodiments, the immune suppressor cells are recombinant cells that have been genetically engineered to include an innate immune effector gene.

Immune suppressive cells are immune cells that function primarily to inhibit an immune response. Examples of immune suppressor cells include immature dendritic cells, Th2 cells, Th3 cells, myeloid-derived suppressor cells, M2 macrophages, regulatory T cells, N2 neutrophils, and infiltration by mesenchymal stem cells possessing immune suppressive properties, are all measurements for selecting patients with immune suppression. In some embodiments, the immune suppressor cell is a regulatory T-cell or a myeloid-derived suppressor cell.

Myeloid-derived suppressor cells (MDSC) are a heterogeneous group of immune cells from the myeloid lineage (i.e., from bone marrow stem cells). Zhao et al., Oncoimmunology, 5 (2), c1004983 (2016). MDSCs expand under pathologic conditions such as chronic infection and cancer, or as a result of altered hematopoiesis. MDSCs differ from other myeloid cell types in that they have immunosuppressive activities, as opposed to immune-stimulatory properties. MDSC activity was originally described as suppressors of T cells, in particular of CD8+ T-cell responses. The spectrum of action of MDSC activity also encompasses NK cells, dendritic cells and macrophages. The myeloid-derived suppressor cells can be allogenic or autologous cells. In some embodiments the myeloid-derived suppressor cells are mammalian myeloid-derived suppressor cells.

Regulator T-cells (Tregs) are a specialized subpopulation of T cells that act to suppress immune response, thereby maintaining homeostasis and self-tolerance. It has been shown that Tregs are able to inhibit T cell proliferation and cytokine production and play a critical role in preventing autoimmunity. Kondelková et al., Acta Medica (Hradec Kralove), 53 (2): 73-7 (2010).

The present invention provides an immune suppressor cell that has been modified to include an innate immune effector. Innate immune effectors are molecules (e.g., peptides) that stimulate the innate immune system. The innate immune system is a relatively non-specific part of the immune system that responds to germs and other foreign substances entering the body. The innate immune system includes defensins, pathogen-associated immunostimulants, ligands effecting TNF (e.g., death receptors and TRAIL), the complement system, macrophages, and natural killer cells. Examples of pathogen-associated immunostimulants include Toll-like receptors, stimulator of interferon gene ligands, retinoic acid-inducible gene 1-like receptor ligands, and C-type lectin receptor ligands.

In some embodiments, the innate immune effector is a ligand effecting a death receptor. Death receptors are members of the TNF receptor superfamily that contain a death domain, such as TNFR1, Fas receptor, DR4 and DR5 and play an important role in apoptosis. The tumor necrosis factor receptor superfamily (TNFRSF) is a group of cytokine receptors characterized by the ability to bind tumor necrosis factors (TNFs) via an extracellular cysteine-rich domain, and are known to those skilled in the art.

In some embodiments, the innate immune effector is TRAIL. See Falschlehner et al., Immunology, 127 (2), 145-154 (2009). TNF-related apoptosis-inducing ligand (TRAIL, also known as Apo2L) interacts with cell surface death receptors (e.g., DR4 and DR5) on cancer cells and activates caspases and other enzymes involved in the apoptotic pathways in cancer cells, but not in healthy cells. Ashkenazi, A., Nat Rev Cancer 2 (6): 420-430 (2002). This specificity makes TRAIL an attractive target for cancer therapy by restoring endogenous death pathways and thus driving cancer cells into self-destruction. TRAIL can bind to several receptors, including TRAIL-R1, TRAIL-R2, TRAIL-R3, and TRAIL-R4. Binding to the first two of these receptors stimulates apoptosis. TRAIL is expressed by a variety of cells of the innate immune system, and can be upregulated by lipopolysaccharide and interferon-β. The amino acid sequence of human TRAIL is known. See Sayed et al., J. Vet. Med. Sci. 66:643-650 (2004) and SEQ ID NO: 1. Recombinant TRAIL is commercially available. See R&D Systems™ Recombinant Human TRAIL/TNSF10, Catalog Number 375-TL/CF.

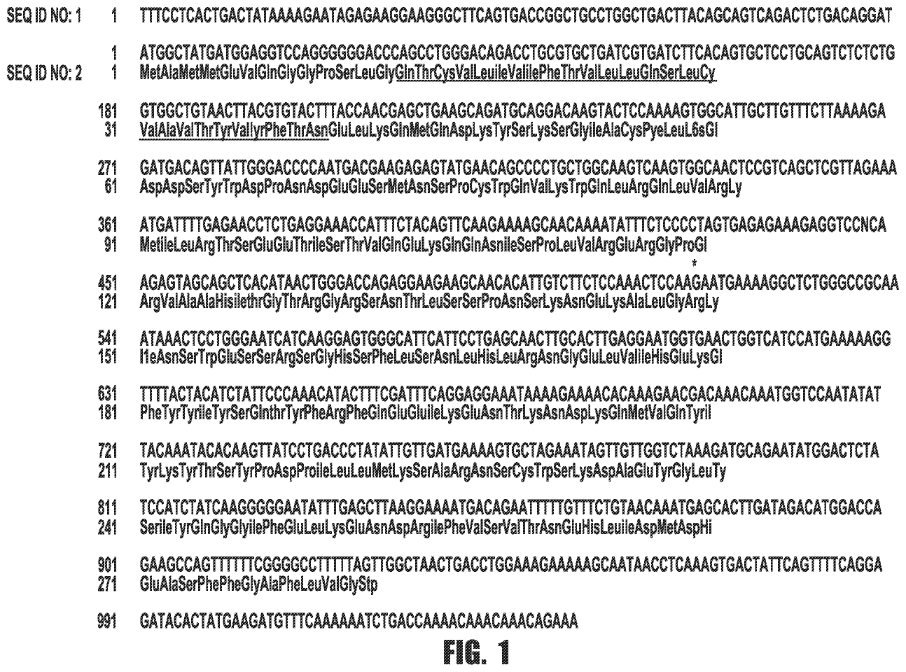

In some embodiments, TRAIL refers to a polypeptide sequence which includes amino acid residues 114-281, inclusive, 95-281, inclusive, residues 92-281, inclusive, residues 91-281, inclusive, residues 41-281, inclusive, residues 15-281, inclusive, or residues 1-281, inclusive, of the amino acid sequence shown in FIG. 1 (SEQ ID NO:1), as well as biologically active fragments, deletional, insertional, or substitutional variants of the above sequences. In one embodiment, the polypeptide sequence comprises residues 114-281 of FIG. 1 (SEQ ID NO:1), and optionally, consists of residues 114-281 of FIG. 1 (SEQ ID NO:1). Optionally, the polypeptide sequence comprises residues 92-281 or residues 91-281 of FIG. 1 (SEQ ID NO:1).

The TRAIL polypeptides may be encoded by the native nucleotide sequence shown in FIG. 1 (SEQ ID NO:2). Optionally, the codon which encodes residue Prol19 (FIG. 1; SEQ ID NO:2) may be “CCT” or “CCG”. In other embodiments, the fragments or variants are biologically active and have at least about 80% amino acid sequence identity, more preferably at least about 90% sequence identity, and even more preferably, at least 95%, 96%, 97%, 98%, or 99% sequence identity with any one of the above recited TRAIL sequences. Optionally, the TRAIL polypeptide is encoded by a nucleotide sequence which hybridizes under stringent conditions with the encoding polynucleotide sequence provided in FIG. 1 (SEQ ID NO:2).

The term “TRAIL extracellular domain” or “TRAIL ECD” refers to a form of TRAIL which is essentially free of transmembrane and cytoplasmic domains. Ordinarily, the ECD will have less than 1% of such transmembrane and cytoplasmic domains, and preferably, will have less than 0.5% of such domains. It will be understood that any transmembrane domain(s) identified for the polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified. In preferred embodiments, the ECD will consist of a soluble, extracellular domain sequence of the polypeptide which is free of the transmembrane and cytoplasmic or intracellular domains (and is not membrane bound). Particular extracellular domain sequences of TRAIL are described in PCT Publication Nos. WO97/01633 and WO97/25428.

In some embodiments, a functional-conservative derivative of the innate immune effector (e.g., TRAIL) is used. Functional-conservative derivatives or variants may result from modifications and changes that may be made in the structure of a polypeptide (and in the DNA sequence encoding it), and still obtain a functional molecule with desirable characteristics (e.g., tumoricidal and/or immunostimulatory effects). Functional-conservative derivatives may also consist of a fragment of the innate immune effector that retains its functionality.

Functional-conservative derivatives or variants are those in which a given amino acid residue in a protein has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A functional-conservative derivative also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared. Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical). Preferably, the similar or homologous sequences are identified by alignment using, for example, the GCG (Genetics Computer Group, Program Manual for the GCG Package, Version 7, Madison, Wisconsin) pilcup program, or any of sequence comparison algorithms such as BLAST, FASTA, etc.

In some embodiments, the immune suppressor cell has been transformed to include an nucleotide sequence. Transformation means introducing DNA into an organism so that the DNA is replicable, either as an extrachromosomal element or by chromosomal integrant. Depending on the host cell used, transformation is done using standard techniques appropriate to such cells. For mammalian cells, the calcium phosphate precipitation method (Graham and van der Eb, Virology 1978, 52:456-457) may be employed. General aspects of mammalian cell host system transformations have been described in U.S. Pat. No. 4,399,216.

A nucleic acid encoding the innate immune effector (e.g., TRAIL) can be cloned or amplified by in vitro methods, such as the polymerase chain reaction (PCR), the ligase chain reaction (LCR), the transcription-based amplification system (TAS), the self-sustained sequence replication system (3SR) and the QP replicase amplification system (QB). The nucleic acid sequence for human TRAIL is known. See U.S. Pat. No. 7,741,282, the disclosure of which is incorporated herein by reference. For example, a polynucleotide encoding the polypeptide can be isolated by polymerase chain reaction of cDNA using primers based on the DNA sequence of the molecule. A wide variety of cloning and in vitro amplification methodologies are well known to persons skilled in the art.

The modified immune suppressor cells of the invention are typically ex vivo cultured cells that have been recombinantly modified to express an innate immune effector. The modified immune suppressor cells may comprise a polynucleotide (such as an expression vector) that encodes an innate immune effector or an active fragment thereof. The immune suppressor cells may be transformed or transfected with such a vector. A vector in the cells may comprise an expression construct that encodes an innate immune vector. A single vector may comprise an expression construct that encodes the innate immune effector, or multiple vectors may comprise expression constructs that encode a plurality of innate immune effectors.

The innate immune effector constructs can be introduced as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc. as appropriate. The construct(s) once completed and demonstrated to have the appropriate sequences may then be introduced into the host cell by any convenient means.

The innate immune effector gene (e.g., TRAIL gene) can be operably linked with an expression control sequence that has a regulatory element such as a promoter (constitutive or regulatable) to drive transgene expression and a polyadenylation sequence downstream of the nucleic acid. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA and stop codons.

Nucleic acids are “operably linked” when they are placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources).

The constructs may be integrated and packaged into non-replicating, defective viral genomes like lentivirus, Adenovirus, Adeno-associated virus (AAV), or Herpes simplex virus (HSV) or others, including retroviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

The DNA encoding innate immune effectors (e.g., TRAIL) may be obtained from any cDNA library prepared from tissue believed to possess the innate immune effector mRNA and to express it at a detectable level. Accordingly, human TRAIL DNA can be conveniently obtained from a cDNA library prepared from human tissues, such as the bacteriophage library of human placental cDNA as described in PCT Publication WO97/25428. The innate immune effector-encoding gene may also be obtained from a genomic library or by oligonucleotide synthesis.

Libraries can be screened with probes (such as antibodies to the innate immune effector (e.g., TRAIL) or oligonucleotides of at least about 20-80 bases) designed to identify the gene of interest or the protein encoded by it. Screening the cDNA or genomic library with the selected probe may be conducted using standard procedures (Sambrook et al., Molecular Cloning: A Laboratory Manual; New York: Cold Spring Harbor Laboratory Press, 1989). An alternative means to isolate the gene encoding the innate immune effector (e.g., TRAIL) is to use PCR methodology (Dieffenbach et al., PCR Primer: A Laboratory Manual, Cold Spring Harbor Laboratory Press, 1995).

Amino acid sequence fragments or variants of innate immune effectors (e.g., TRAIL) can be prepared by introducing appropriate nucleotide changes into the innate immune effector DNA, or by synthesis of the desired innate immune effector polypeptide. Such fragments or variants represent insertions, substitutions, and/or deletions of residues within or at one or both of the ends of the intracellular region, the transmembrane region, or the extracellular region, or of the amino acid sequence shown for the full-length TRAIL in FIG. 1 (SEQ ID NO:1). Any combination of insertion, substitution, and/or deletion can be made to arrive at the final construct, provided that the final construct possesses, for instance, a desired biological activity or apoptotic activity as defined herein. In a preferred embodiment, the fragments or variants have at least about 80% amino acid sequence identity, more preferably, at least about 90% sequence identity, and even more preferably, at least 95%, 96%, 97%, 98% or 99% sequence identity with, for example, the sequences identified herein for the intracellular, transmembrane, or extracellular domains of innate immune effector (e.g., TRAIL), or the full-length sequence for innate immune effector (e.g., TRAIL). The amino acid changes also may alter post-translational processes of the, such as changing the number or position of glycosylation sites or altering the membrane anchoring characteristics.

### Treatment of Cancer Using Modified Immune Suppressor Cells

In another aspect, the present invention provides methods for treating cancer in a subject, comprising administering an effective amount of an immune suppressor cell modified to include an innate immune effector to the subject. In some embodiments, the immune suppressor cell is a regulatory T-cell or a myeloid-derived suppressor cell, while in further embodiments the innate immune effector is TRAIL.

Cancer is a disease of abnormal and excessive cell proliferation. Cancer is generally initiated by an environmental insult or error in replication that allows a small fraction of cells to escape the normal controls on proliferation and increase their number. The damage or error generally affects the DNA encoding cell cycle checkpoint controls, or related aspects of cell growth control such as tumor suppressor genes. As this fraction of cells proliferates, additional genetic variants may be generated, and if they provide growth advantages, will be selected in an evolutionary fashion. Cells that have developed growth advantages but have not yet become fully cancerous are referred to as precancerous cells. Cancer results in an increased number of cancer cells in a subject. These cells may form an abnormal mass of cells called a tumor, the cells of which are referred to as tumor cells. The overall amount of tumor cells in the body of a subject is referred to as the tumor load. Tumors can be either benign or malignant. A benign tumor contains cells that are proliferating but remain at a specific site and are often encapsulated. The cells of a malignant tumor, on the other hand, can invade and destroy nearby tissue and spread to other parts of the body through a process referred to as metastasis.

Cancer is generally named based on its tissue of origin. There are several main types of cancer. Carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma and multiple myeloma are cancers that begin in the cells of the immune system. In some embodiments, the cancer is selected from the group of cancer types consisting of sarcoma, carcinoma, and lymphoma.

Cancer can also be characterized based on the organ in which it is growing. Examples of cancer characterized in this fashion include bladder cancer, prostate cancer, liver cancer, breast cancer, colon cancer, and leukemia. Solid tumors are a solid mass of cancer cells that grow in organ systems, as understood by those skilled in the art, and are more associated with the formation of an immune suppressive tumor microenvironment. In some embodiments, the cancer being treated a solid tumor cancer selected from the group consisting of breast, colon, bladder, prostate, and lung cancer.

In some embodiments, the cancer being treated is a drug-resistant cancer. A variety of characteristics of cancer cells can make them drug resistant. Vasan et al., Nature volume 575, p. 299-309 (2019). One of the factors associated with drug-resistance is the creation of an immune suppressive tumor microenvironment. The tumor microenvironment (TME) facilitates growth of tumor cells via the interplay between tumor cells and surrounding cells. TME contains a variety of cells, including fibroblasts, endothelial cells, mast cells, and immune cells. Fibroblasts are predominantly related to vascular development and tumor cell proliferation, while endothelial cells are deemed to be an important source of cytokines that can boost TME homeostasis. Immune cells in the TME include T lymphocytes (CD4+T lymphocytes and CD8+T lymphocytes), natural killer (NK) cells, tumor-associated macrophages (TAM), dendritic cells (DC), myeloid-derived suppressor cells (MDSC), regulatory T cells (Treg), and neutrophils. Parente et al., Gastroenterol Res Pract, 2018:7530619 (2018). The population of immune cells in the TME differs from that found in healthy tissue and contributes to the formation of an immune suppressive tumor microenvironment.

The modified immune suppressor cells can be used for both prophylactic and therapeutic treatment. The modified immune suppressor cells can, for example, be administered prophylactically to a mammal prior to the development of cancer. Prophylactic administration, also referred to as prevention, is effective to decrease the likelihood that cancer will develop in the subject. For prophylactic treatment, the subject is any human or animal subject, and preferably is a human subject who is at risk of acquiring a disorder characterized by unwanted, rapid cell proliferation, such as cancer, or is suspected of having cancer.

A human in need thereof may be an individual who has or is suspected of having a cancer. In some of variations, the human is at risk of developing a cancer (e.g., a human who is genetically or otherwise predisposed to developing a cancer) and who has or has not been diagnosed with the cancer. As used herein, an “at risk” subject is a subject who is at risk of developing cancer (e.g., a hematologic malignancy). The subject may or may not have detectable disease, and may or may not have displayed detectable disease prior to the treatment methods described herein. An at-risk subject may have one or more so-called risk factors, which are measurable parameters that correlate with development of cancer, such as described herein. A subject having one or more of these risk factors has a higher probability of developing cancer than an individual without these risk factor(s). These risk factors may include, for example, age, sex, race, diet, history of previous disease, presence of precursor disease, genetic (e.g., hereditary) considerations, and environmental exposure. In some embodiments, a human at risk for cancer includes, for example, a human whose relatives have experienced this disease, and those whose risk is determined by analysis of genetic or biochemical markers. Prior history of having cancer may also be a risk factor for instances of cancer recurrence. In some embodiments, provided herein is a method for treating a human who exhibits one or more symptoms associated with cancer (e.g., a hematologic malignancy).

In one embodiment of the invention patients are chosen who have a high degree of cancer-associated immune suppression. Immune suppression is assessed using different means known in the art and can include quantification of number of immune cells in circulation, quantification of activity of immune cells in circulation, quantification of the number of immune cells found intratumorally, quantification of activity of immune cells found intratumorally, quantification of the number of immune cells found peritumorally, and quantification of activity of immune cells found peritumorally. Immune suppression is more commonly found in solid tumors. In some embodiments of the invention, quantification of immune cells comprises identification and assessment of activity of cells possessing tumor cytolytic and/or tumor inhibitory activity, such cells include natural killer cells (NK), gamma delta T cells, natural killer T cells (NKT), innate lymphoid cells, cytotoxic T lymphocytes (CTL), and helper T cells (Th). Activities of immune cells could be ability to stimulate other immune cells, killing activity, tumor-growth inhibitory activity, as well as suppression of angiogenesis. Other means of assessing suppression of immunity includes quantification of immune suppressive cells. For example, elevations in immature dendritic cells, Th2 cells, Th3 cells, myeloid suppressor cells, M2 macrophages, T regulatory cells, N2 neutrophils, and infiltration by mesenchymal stem cells possessing immune suppressive properties, are all measurements for selecting patients with immune suppression.

Alternatively, the modified immune suppressor cells can be administered therapeutically to a subject that already has cancer. For purposes of treatment, a subject at risk includes any human or animal subject who has a disorder characterized by unwanted, rapid cell proliferation. Such disorders include but are not limited to cancers and precancers. In one embodiment of therapeutic administration, administration of the modified immune suppressor cells is effective to eliminate the cancer; in another embodiment, administration of the modified immune suppressor cells is effective to decrease the symptoms or spread of the cancer.

The effectiveness of cancer treatment may be measured by evaluating a reduction in tumor load or decrease in tumor growth in a subject in response to the administration of the modified immune suppressor cells. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

Methods of cancer treatment using modified immune suppressor cells can further include the step of ablating the cancer. Ablating the cancer can be accomplished using a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, and administration of immunotoxins. The use of an additional method of cancer treatment can be used before, during, or after treatment with modified immune suppressor cells.

Because of the importance of the immune suppressive tumor environment, in some embodiments the method of cancer treatment can further include immunotherapy. For example, in some embodiments, the method of treating cancer further comprises immune checkpoint therapy or administering antibodies (e.g., bispecific antibodies). Antibodies can include monoclonal antibodies used for immunotherapy. Antibodies also include bispecific antibodies (BsAbs). which are antibodies with two binding sites directed at two different antigens or two different epitopes on the same antigen. The clinical therapeutic effects of BsAbs are superior to those of monoclonal antibodies, with broad applications for tumor immunotherapy. Ma et al., Front Immunol., 12:626616 (2021).

In some embodiments, an immune suppressor cell modified to include an innate immune effector may be used together with checkpoint inhibitor drugs. For example, administration of an effective amount of modified immune suppressor cells can be used to increase the effectiveness of immune checkpoint therapy. Examples of such checkpoint inhibitors include: a) Inhibitors of Programmed Death 1 (PD-1, CD279), such as nivolumab (OPDIVO®, BMS-936558, MDX1106, or MK-34775), and pembrolizumab (KEYTRUDA®, MK-3475, SCH-900475, lambrolizumab, CAS Reg. No. 1374853-91-4), as well as the PD-1 blocking agents described in U.S. Pat. Nos. 7,488,802, 7,943,743, 8,008,449, 8,168,757, 8,217,149, WO 03042402, WO 2008156712, WO 2010089411, WO 2010036959, WO 2011066342, WO 2011159877, WO 2011082400, and WO 2011161699; b) Inhibitors of Programmed Death-Ligand 1 (PD-L1 also known as B7-H1 and CD274), including antibodies such as BMS-936559, MPDL3280A), MEDI14736, MSB0010718C, and MDX1105-01); also including: atezolizumab, durvalumab and avelumab; c) Inhibitors of CTLA-4, such as ipilimumab (YERVOY®, MDX-010, BMS-734016, and MDX-101), tremelimumab, antibody clone BNI3 (Abcam), RNA inhibitors, including those described in WO 1999/032619, WO 2001/029058, U.S. 2003/0051263, U.S. 2003/0055020, U.S. 2003/0056235, U.S. 2004/265839, U.S. 2005/0100913, U.S. 2006/0024798, U.S. 2008/0050342, U.S. 2008/0081373, U.S. 2008/0248576, U.S. 2008/055443, U.S. Pat. Nos. 6,506,559, 7,282,564, 7,538,095 and 7,560,438 (each incorporated herein by reference); d) inhibitors of PD-L2 (B7-DC, CD273), such as AMP-224 (Amplimune, Inc.) and rHlgM12B7; and c) Inhibitors of checkpoint proteins, including: LAG3, such as IMP321; TIM3 (HAVCR2); 2B4; A2aR, ID02; B7H1; B7-H3 or B7H3, such as antibody MGA271; B7H4; BTLA; CD2; CD20, such as ibriturmomab tiuxetan, ofatumumab, rituximab, obinutuzumab and tositumomab; CD27, such as CDX-1127; CD28; CD30, such as brentuximab vedotin; CD33, such as gemtuzumab ozogamicin; CD40; CD52, such as alemtuzumab; CD70; CD80; CD86; CD112; CD137; CD160; CD226; CD276; DR3; OX-40 (TNFRSF.sub.4 and CD134); GAL9; GITR; such as TRX518; HAVCR2; HVEM; IDO1; ICOS (inducible T cell costimulator; CD278); such as MEDI570 (MedImmune LLC) and AMG557 (Amgen); KIR; LAIR; LIGHT; MARCO (macrophage receptor with collagencous structure); PS (phosphatidylserine); SLAM; TIGIT; VISTA; and VTCNI; or a combinations thereof. In another variation, the checkpoint inhibitor is an inhibitor of a checkpoint protein selected from the group of PD-1, PD-L1 and CTLA-4. In another variation, the checkpoint inhibitor is selected from the group of an anti-PD-1 antibody, and anti-PD-L1 antibody, and an anti-CTLA-4 antibody. In one variation, the anti-PD-1 antibody is selected from the group of nivolumab, pembrolizumab, and lambrolizumab. In another variation, the anti-PD-L1 antibody is selected from the group of as BMS-936559, MPDL3280A, MEDI4736, MSB0010718C, and MDX1105-01. In yet other variations, the anti-PD-L1 antibody is selected from the group of durvalumab, atezolizumab, and avelumab. In another variation, the anti-CTLA-4 antibody is selected from the group of ipilimumab and tremelimumab. In one embodiment, the check point inhibitor is selected from the group consisting of nivolumab, pembrolizumab, lambrolizumab, BMS-936559, MPDL3280A, MEDI4736, MSB0010718C, MDX1105-01, durvalumab, atezolizumab, avelumab, ipilimumab, and tremelimumab. In certain embodiment, the check point inhibitor is selected from the group consisting of nivolumab, pembrolizumab, lambrolizumab, durvalumab, atezolizumab, avelumab, ipilimumab, and tremelimumab. In one embodiment, the check point inhibitor is selected from the group consisting of nivolumab, pembrolizumab, durvalumab, atezolizumab, and avelumab.

### Administration and Formulation

The modified immune suppressor cells should be administered and dosed in accordance with good medical practice, taking into account the site and method of administration, scheduling of administration, patient age, sex, body weight, the nature and severity of the disorder to be treated or prevented, and other factors known to medical practitioners. The cells may be administered in a single dose or in divided doses. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art.

The number of cells that are employed will depend upon a number of circumstances, the purpose for the introduction, the lifetime of the cells, the protocol to be used, for example, the number of administrations, the ability of the cells to multiply, the stability of the recombinant construct, and the like. Typically said dose is about 10×10⁶ cells/kg of subject weight or lower, is about 9×10⁶ cells/kg or lower, is about 8×10⁶ cells/kg or lower, is about 7×10⁶ cells/kg or lower, is about 6×10⁶ cells/kg or lower, is about 5×10⁶ cells/kg or lower. In an alternative embodiment said dose may be between about 0.25×10⁶ cells/kg to about 5×10⁶ cells/kg; or more preferably about 1×10⁶ cells/kg to about 5×10⁶ cells/kg. Accordingly in further alternative embodiments the dose may be about 0.25×10⁶ cells/kg, 0.5×10⁶ cells/kg, 0.6×10⁶ cells/kg, 0.7×10⁶ cells/kg; 0.8×10⁶ cells/kg; 0.9×10⁶ cells/kg; 1.1×10⁶ cells/kg; 1.2×10⁶ cells/kg; 1.3×10⁶ cells/kg; 1.4×10⁶ cells/kg; 1.5×10⁶ cells/kg; 1.6×10⁶ cells/kg; 1.7×10⁶ cells/kg; 1.8×10⁶ cells/kg; 1.9×10⁶ cells/kg or 2×10⁶ cells/kg. The dose may, in other embodiments, be between 0.1 and 1 million cells/kg; or between 1 and 2 million cells/kg; or between 2 and 3 million cells/kg; or between 3 and 4 million cells/kg; or between 4 and 5 million cells/kg; or between 5 and 6 million cells/kg; or between 6 and 7 million cells/kg; or between 7 and 8 million cells/kg; or between 8 and 9 million cells/kg; or between 9 and 10 million cells/kg.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection. In some embodiments, the modified immune suppressor cells are administered directly to the tumor by injection.

Modified immune suppressor cells can be supplied in the form of a pharmaceutical composition, comprising an isotonic excipient prepared under sufficiently sterile conditions for human administration. The composition can be sterile. The formulation should suit the mode of administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising a population of modified immune suppressor cells will be adapted in accordance with the route and device used for administration.

In some embodiments, the modified immune suppressor cells are administered together with a pharmaceutically acceptable carrier. Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets.

Preferably, the cells are administered by injection, e.g., intravenously. The pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An immune suppressor cell modified to include an innate immune effector.

2. The immune suppressor cell of claim 1, wherein the immune suppressor cell is a regulatory T-cell or a myeloid-derived suppressor cell.

3. The immune suppressor cell of claim 1, wherein the innate immune effector is TRAIL.

4. The immune suppressor cell of claim 3, wherein the TRAIL comprises an amino acid sequence having 95% identity to SEQ ID NO: 1.

5. The immune suppressor cell of claim 3, wherein the immune suppressor cell has been transformed to include a nucleotide sequence having 95% identity to SEQ ID NO: 2 that is operably linked to a promoter.

6. A method of treating cancer in a subject, comprising administering an effective amount of an immune suppressor cell modified to include an innate immune effector to the subject.

7. The method of claim 6, wherein the immune suppressor cell is a regulatory T-cell or a myeloid-derived suppressor cell.

8. The method of claim 6, wherein the innate immune effector is TRAIL.

9. The immune suppressor cell of claim 8, wherein the TRAIL comprises an amino acid sequence having 95% identity to SEQ ID NO: 1.

10. The immune suppressor cell of claim 8, wherein the immune suppressor cell has been transformed to include a nucleotide sequence having 95% identity to SEQ ID NO: 2 that is operably linked to a promoter.

11. The method of claim 6, wherein the cancer is selected from the group of cancer types consisting of sarcoma, carcinoma, and lymphoma.

12. The method of claim 6, wherein the cancer is a solid tumor cancer selected from the group consisting of breast, colon, bladder, prostate, and lung cancer.

13. The method of claim 6, wherein the cancer is a drug-resistant cancer.

14. The method of claim 6, wherein an additional type of cancer therapy is used to treat the subject.

15. The method of claim 6, wherein the method of treating cancer further comprises immune checkpoint therapy or administration of antibodies.

16. The method of claim 6, wherein the modified immune suppressor cells are administered directly to the tumor by injection.


SEQUENCE LISTING (2 sequences)

SEQ ID NO: 1 - protein, 843 aa, Homo sapiens
METALAMETMETGLUVALGLNGLYGLYPROSERLEUGLYGLNTHRCYSVALLEUILEVALILEPHETHRVALLEULEUGLNSERLEUCYSVALALAVALTHRTYRVALTYRPHETHRASNGLULEULYSGLNMETGLNASPLYSTYRSERLYSSERGLYILEALACYSPHELEULYSGLUASPASPSERTYRTRPASPPROASNASPGLUGLUSERMETASNSERPROCYSTRPGLNVALLYSTRPGLNLEUARGGLNLEUVALARGLYSMETILELEUARGTHRSERGLUGLUTHRILESERTHRVALGLNGLULYSGLNGLNASNILESERPROLEUVALARGGLUARGGLYPROGLNARGVALALAALAHISILETHRGLYTHRARGGLYARGSERASNTHRLEUSERSERPROASNSERLYSASNGLULYSALALEUGLYARGLYSILEASNSERTRPGLUSERSERARGSERGLYHISSERPHELEUSERASNLEUHISLEUARGASNGLYGLULEUVALILEHISGLULYSGLYPHETYRTYRILETYRSERGLNTHRTYRPHEARGPHEGLNGLUGLUILELYSGLUASNTHRLYSASNASPLYSGLNMETVALGLNTYRILETYRLYSTYRTHRSERTYRPROASPPROILELEULEUMETLYSSERALAARGASNSERCYSTRPSERLYSASPALAGLUTYRGLYLEUTYRSERILETYRGLNGLYGLYILEPHEGLULEULYSGLUASNASPARGILEPHEVALSERVALTHRASNGLUHISLEUILEASPMETASPHISGLUALASERPHEPHEGLYALAPHELEUVALGLY

SEQ ID NO: 2 - DNA, 1042 bp, Homo sapiens
  source 1..1042: mol_type = genomic DNA
tttcctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtccncagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaa